(19) United States
(12) Patent Application Publication
(10) Pub. No.: US 2026/0240894 A1
(43) Pub. Date: Aug. 20, 2026

(54) IMPROVING LAYER BIRD PERFORMANCE USING AN OLIGOSACCHARIDE PREPARATION

(71) Applicant: DSM IP ASSETS B.V., Heerlen (NL)

(72) Inventors: Elkin AMAYA, Getzersdorf (AT); Fernando CISNEROS GONZALEZ, Getzersdorf (AT); Elisa FOLEGATTI, Getzersdorf (AT); Phokela Jonathan SEGOBOLA, Getzersdorf (AT); Murtala UMAR FARUK, Getzersdorf (AT)

(21) Appl. No.: 18/843,865

(22) PCT Filed: Mar. 7, 2023
(86) PCT No.: PCT/EP2023/055722

§ 371 Date: Sep. 4, 2024

(30) Foreign Application Priority Data
Mar. 9, 2022 (EP) 22161149.4

(51) Int. Cl. A61K 31/715; A23K 20/163; A23K 50/75; A61P 3/00; A61P 31/04

(52) U.S. Cl. CPC A61K 31/715; A23K 20/163; A23K 50/75; A61P 3/00; A61P 31/04

(57) ABSTRACT

The present invention relates to a method for improving performance of a layer bird by administering a nutritional composition comprising an oligosaccharide preparation to the layer bird, and to a use of an oligosaccharide preparation to improve performance of a layer bird.

The present invention relates to a method for improving performance of a layer bird and to the use of an oligosaccharide preparation.

Throughout all agricultural fields, pressure is increased to provide more product at lower costs. At the same time, product quality must not suffer but should instead be increased. This same trend holds true for commercial egg production.

Due to the aforementioned pressure for more efficient production, poultry farmers select bird breeds which have been identified to show an optimized performance for the respective purpose. Accordingly, chicken breeds particularly bred for the purpose of broiler production are commercially available as well as for egg production. Examples of breeds that have been selected for optimized performance in commercial egg production, are Isa White, Isa Brown, White Leghorns, Rhode Island Red, Golden Comet, Speckled Sussex etc. Such breeds which have been bred particularly for the purpose of increased egg laying performance are referred to as layer birds or laying birds (or sometimes as “layers” in short). Driven by market demand, chicken layers, laying hens, are currently the dominant species in commercial egg production.

As a consequence of the differing purposes, i.e. meat production vs. egg production, as well as of the individual needs of the broiler breeds compared to the layer breeds, farming regimes, in particular with regard to dieting have been finetuned for optimized performance.

Despite these efforts in breeding and animal husbandry, there is still a need for further performance improvements. It is thus an objective of the present invention to provide a way to improve the performance of layer birds.

This objective has been found to be achieved by a method for improving performance (e.g. increased body weight gain, increased laying index, increased egg mass, increased percentage of XL eggs, reduced feed conversion per kg eggs and/or reduced feed conversion per dozen eggs) of a layer bird (e.g. layer poultry, layer chicken, layer hen), comprising the steps of a) providing a layer bird; b) providing a nutritional composition suitable for layer birds, said nutritional composition comprising an oligosaccharide preparation (e.g. a synthetic oligosaccharide preparation), wherein the oligosaccharide preparation comprises at least n fractions of oligosaccharides each having a distinct degree of polymerization selected from 1 to n (DP1 to DPn fractions), wherein n is an integer greater than or equal to 2, and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; and c) administering the nutritional composition to the layer bird.

In some embodiments of the method described herein, the performance of the layer bird is improved in terms of improved nitrogen utilization. A nitrogen utilization is considered improved, when more egg mass is produced per mass of ingested nitrogen, compared to a control layer bird that was not used in or subjected to the method described herein.

In some embodiments of the method described herein, the performance of the layer bird is improved in terms of nutrient digestibility. Nutrient digestibility is considered improved, when more body weight (or mass) and/or more egg mass is produced per mass of ingested nutrients, compared to a control layer bird that was not used in or subjected to the method described herein.

In some embodiments of the method described herein, the performance of the layer bird is improved in terms of resilience to enteric stress. Enteric stress such as E. coli and Salmonella infection are known to cause layer birds to show weight loss, decreased weight gain, and/or decreased conversion of feed into egg mass or egg amount. A layer bird is considered to be more resilient to enteric stress, when said layer bird shows no or less weight loss, no or less decrease in weight gain, and/or no or less decrease in conversion of feed into egg mass or egg amount, compared to a control layer bird that was not used in or subjected to the method described herein. Experimentally, enteric stress can be intentionally imposed on layer birds in challenged studies e.g. by way of Salmonella and/or E. coli infection (Adhikari et al. 2018. Poult Sci. 97(7):2525-2533). E. coli control is particularly important in the animal intestine, Salmonella control in birds and eggs. Salmonella is an important public risk for egg consumers. Salmonella are Gram-negative bacteria that can cause an illness called salmonellosis in humans. Salmonella is commonly found in the intestines of healthy birds and mammals. In some embodiments of the method described herein, the performance of the layer bird is improved in terms of a reduced amount of produced eggs contaminated with Salmonella.

Surprisingly, performing the method described herein was found to result inter alia in a significantly increased final body weight as well as body weight gain of layer birds to which the nutritional composition comprising the oligosaccharide preparation described herein was administered, compared to layer birds to which a control nutritional composition was administered, which control nutritional composition did not comprise the oligosaccharide preparation. In addition, significantly increased laying index, increased egg mass and increased percentage of XL eggs, and reduced feed conversion per kg and per dozen eggs was found by layer birds to which the nutritional composition comprising the oligosaccharide preparation described herein was administered, compared to layer birds to which the control nutritional composition was administered.

Merely for clarification, the improvement(s) in performance of the layer bird administered the nutritional composition comprising the oligosaccharide composition, are compared to a control layer bird of the same breed, to which control layer bird a control nutritional composition is administered that only differs from the nutritional composition mentioned above in that the control nutritional composition does not comprise the oligosaccharide composition.

As described herein, a layer bird can be any bird breed that has been bred and thus evolutionary redesigned by human towards improved egg laying performance. The layer bird may be layer poultry, e.g. chicken, duck, goose, turkey, guinea fowl, pigeon or quail. In particular, a layer bird can be a layer chicken. Non-limiting examples for layer chicken breeds are Isa White, Isa Brown, (White) Leghorns, Rhode Island Red, Golden Comet, (Speckled) Sussex, Babcock, Bovans, Dekalb, Hisex, Shaver, Warren, Azur, Noirans, Olive, (Barred) Plymouth Rock, Ancona, Barnevelder, Hamburg, Marans, Buff Orpington, Easter Eggers, Ameraucana, Australorp, Delaware, Euskal oils, Faverolles, Golden laced wyandotte, Jamerson, New Hampshire red, Red sex link, Welsummer, Austrawhite, (Golden Laced) Wyandotte, Minorca, Lohmann Brown, Penedesenca, La Bresse etc.

Administering the nutritional composition as referred to in step c) of the method described herein is intended to be construed as providing the nutritional composition to the layer bird for consumption by said layer bird. In other words, the nutritional composition is fed to the layer bird.

In one embodiment, this administering/providing/feeding may be effected by providing the nutritional composition to the layer bird in an ad libitum feeding fashion. Preferably, the oligosaccharide preparation of the method described herein is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed), more preferably of at least 100 g, even more preferably of at least 500 g, or even more e.g. at least 900 g per ton of feed.

In some embodiments, administering the nutritional composition comprises providing the nutritional composition to an animal such that the animal may ingest the nutritional composition at will. In such embodiments, the animal ingests at least some portion of the nutritional composition.

Also, the nutritional composition may be provided to the animal on any appropriate schedule. In some embodiments, the animal is provided the nutritional composition on a daily basis, on a weekly basis, on a monthly basis, on an every other day basis, for at least three days out of every week, or for at least seven days out of every month.

In some embodiments, the nutritional composition is administered to the animal multiple times in a day. For examples, in some embodiments, the nutritional composition is administered to the animal at least 1, 2, 3, 4, 5, 6, 7, 8, 9, or 10 times a day. In some embodiments, the nutritional composition is administered to the animal at most 1, 2, 3, 4, 5, 6, 7, 8, 9, or 10 times a day. In some embodiments, the nutritional composition is administered to the animal at least 1, 2, 3, 4, 5, 6, 7, 8, 9, 10, 15, or 20 times a week. In some embodiments, the nutritional composition is administered to the animal at most 1, 2, 3, 4, 5, 6, 7, 8, 9, 10, 15, or 20 times a week. In some embodiments, the nutritional composition is administered to the animal every day, every other day, every 3 days, every 4 days, every week, every other week, or every month.

In certain embodiments, the nutritional composition is administered to the animal at certain time during the day. For example, in certain embodiments, the nutritional composition is administered to the animal in the morning, in the afternoon, and/or in the evening, or any combination thereof.

In some embodiments, the animal is provided the nutritional composition during certain diet phases. For example, some animals are provided a starter diet between 0 to 14 days of age. In other embodiments, an animal is provided a grower diet between 15 to 28 days of age, between 15 to 35 days of age, or between 15 to 39 days of age. In still other embodiments, an animal is provided a finisher diet between 29 to 35 days of age, between 36 to 42 days of age, or between 40 to 46 days of age.

The nutritional composition may be fed to individual animals or an animal population. For example, in some embodiments, where the animal is poultry, the nutritional composition may be fed to an individual poultry or a poultry population.

The nutritional composition may be provided to an animal in any appropriate form, including, for example, in solid form, in liquid form, or a combination thereof. In certain embodiments, the nutritional composition is a liquid, such as a syrup or a solution. In other embodiments, the nutritional composition is a solid, such as pellets or powder. In yet other embodiments, the nutritional composition may be fed to the animal in both liquid and solid components, such as in a mash.

In one embodiment, n of the oligosaccharide preparation of the method described herein is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In another embodiment, at least one fraction of the oligosaccharide preparation of the method described herein comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance

In a further embodiment, the oligosaccharide preparation of the method described herein has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

In one embodiment of the invention, the relative abundance of oligosaccharides in each of the n fractions of the oligosaccharide preparation of the method described herein decreases monotonically with its degree of polymerization.

In one aspect, the invention relates to an egg obtained by the method described herein. Without wishing to be bound by theory, the inventors believe that due to the positive effect of administering the nutritional composition comprising the oligosaccharide preparation described herein to a layer bird, the eggs produced by said layer bird differ from eggs that were produced differently in that the microbiome present on the eggs produced by a method according to the invention comprises more microorganisms that are beneficial for human health than other, regular eggs.

In addition or alternatively, the eggs produced by the method described herein show a significantly increased concentration of one or more of the following components comprised in the egg yolk: thiamin; riboflavin; niacin; vitamin B6; vitamin B12; vitamin A; vitamin E; vitamin D, in particular vitamin D2 and/or D3); vitamin K; calcium; iron; magnesium; phosphorus; potassium; sodium; zinc; carotenoids, in particular lutein, zeaxanthin, apo-ester and/or canthaxanthin.

In some embodiments, the invention relates to an egg obtained by the method described herein, wherein the egg comprises an egg yolk having a higher pigmentation compared to a control egg yolk of a control egg that was obtained from a control layer bird to which a control nutritional composition was administered, which control nutritional composition did not comprise the oligosaccharide preparation referred to herein. In other words, such an egg comprises an egg yolk with more favorable (e.g. a higher) carotenoid content and thus with a more golden color rather than a pale color. In particular, the egg yolk of such an egg comprises an increased concentration of one or more of yellow carotenoids having an absorption maximum at a wavelength from 445 to 450 nm (e.g. lutein, zeaxanthin, apo-ester); and/or an increased concentration of one or more red carotenoids having an absorption maximum at a wavelength from 465 to 470 nm (e.g. canthaxanthin)—compared to the egg yolk of an egg that was produced by a control bird to which a control nutritional composition was administered, which control nutritional composition did not comprise the oligosaccharide preparation referred to herein. Preferably, the egg yolk of the egg according to the invention comprises an increased concentration in one or more yellow carotenoids (e.g. lutein, zeaxanthin, apo-ester) and an increased concentration in one or more red carotenoids (e.g. canthaxanthin). Due to the increased carotenoid content, such eggs have a richer and more diverse nutritional value.

In some embodiments, the invention relates to an egg obtained by the method described herein, wherein the egg has an improved (e.g. increased) eggshell quality (e.g. increased breaking strength).

In some embodiments, the invention relates to an egg obtained by the method described herein, wherein the egg has a lower risk for comprising Salmonella. In some embodiments, the invention relates to a plurality of the eggs according to the invention, which eggs are obtained by the method according to the invention, wherein the number of eggs comprising Salmonella amongst said plurality of eggs is lower compared to a control plurality of eggs which were obtained by a different method, in particular obtained from a bird (in particular a layer bird, e.g. layer poultry, layer chicken, layer hen) to which a control nutritional composition was administered, which control nutritional composition did not comprise the oligosaccharide preparation referred to herein. Merely for clarification, the control nutritional composition referred to herein differs from the nutritional composition suitable for layer birds comprising the oligosaccharide preparation referred to herein only in that the control nutritional composition does not comprise said oligosaccharide preparation.

In a further aspect, the invention relates to a method for improving animal welfare (e.g. improved feather score, improved bumble foot score, and/or improved human-animal relationship as determined by the Avoidance Distance Test (ADT)) of a layer bird (e.g. layer poultry, layer chicken, layer hen), comprising the steps of a) providing a layer bird; b) providing a nutritional composition suitable for layer birds comprising an oligosaccharide preparation (e.g. a synthetic oligosaccharide preparation), wherein the oligosaccharide preparation comprises at least n fractions of oligosaccharides each having a distinct degree of polymerization selected from 1 to n (DP1 to DPn fractions), wherein n is an integer greater than or equal to 2, and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; and c) administering the nutritional composition to the layer bird. Feather score and how to determine it is described in Welfare Quality Network (2019), Welfare Quality assessment protocol for laying hens, version 2.0, ISBN/EAN 978-90-78240-06-8. Bumble foot score and how to determine it is described in Blair 2013, Vet Clin North Am Exot Anim Pract. 16(3):715-35. ADT and how to determine it is described in Meuser et al. 2021. Animals (Basel): 11(3):679.

In a further aspect, the invention relates to an oligosaccharide preparation for use in treatment, amelioration and/or prophylaxis of a layer bird (e.g. layer poultry, layer chicken, layer hen), wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

In a further aspect, the invention relates to an oligosaccharide preparation for use in treatment, amelioration and/or prophylaxis of a layer bird suffering from one or more disorders associated with an irregular level of one or more of the following blood parameters: sodium (Na), potassium (K), glucose, urea nitrogen/urea, creatinine, hematocrit, hemoglobin, chloride, total carbon dioxide (TCO₂), ionized calcium, sodium (Na⁺), potassium (K⁺), aspartate aminotransferase, bile acid(s), creatine kinase, uric acid, phosphorous, calcium, total protein, albumin, globulin; wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

In a further aspect, the invention relates to an oligosaccharide preparation for use in treatment, amelioration and/or prophylaxis of a layer bird suffering from enteric stress (e.g. caused by Salmonella, Escherichia coli and/or coccidia); wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

In another aspect, the invention relates to a use of an oligosaccharide preparation (e.g. synthetic oligosaccharide preparation) to improve the performance (e.g. increased body weight gain, increased laying index, increased egg mass, increased percentage of XL eggs, reduced feed conversion per kg eggs and/or reduced feed conversion per dozen eggs) of a layer bird (e.g. layer poultry, layer chicken, layer hen), wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry. Analogous to the method according to the invention, the improvement(s) in performance of the layer bird administered the nutritional composition comprising the oligosaccharide composition, are compared to a control layer bird of the same breed, which control layer bird is administered a control nutritional composition that only differs from the nutritional composition comprising the oligosaccharide composition (as mentioned above) in that the control nutritional composition does not comprise the oligosaccharide composition.

In one embodiment, the oligosaccharide preparation of the use described herein is administered to the bird before, after, and/or simultaneously with the diet of the layer bird, preferably simultaneously with the diet. At least part of the diet may be a nutritional composition suitable for being administered to a layer bird. In a particular embodiment, the nutritional composition referred to herein may be or comprise a basal diet, wherein said basal diet comprises 31.730% maize, 25.000% wheat, 10.000% sunflower meal, 18.972% soybean meal, 3.989% soy oil, 2.583% calcium carbonate fine, 6.026% calcium carbonate coarse, 0.549% monocalcium phosphate, 0.356% salt, 0.193% DL-methionine, 0.161% L-lysine HCl, 0.029% threonine, 0.007% tryptophane and 0.400% Vit&Min premix; and/or wherein the basal diet has the following calculated analysis: 89.24% dry matter, 11.83% ash, 17.00% crude protein, 5.87% ether extract, 4.48% crude fibre, 11.18% neutral detergent fibre, 35.57% starch, 3.8% calcium, 0.507% total phosphorus, 0.280% phytic phosphorus, 0.380% average phosphorus, 0.160% sodium, 0.296% chloride, 2767 kcal/kg AMEn, 11.58 MJ/kg AMEn, 0.91% total lysine, 0.47% total methionine, 0.77 total methionine+cysteine, 0.64% total threonine, 0.21% total tryptophan, 0.800% dig. lysine, 0.446% dig. methionine, 0.680% dig. methionine+cysteine, 0.180% dig. threonine, 0.605% dig. tryptophan, 0.605% dig. isoleucine, 0.691% dig. valine, 1.011 dig. arginine. Therein, said Vit&Min premix comprises (per kg of the basal diet): Vitamin A (3a672a): 40,000 IU; Vitamin D3 (3a671): 8,000 IU; Vitamin E (α-tocopherol 3a700): 40.0 mg; Vitamin K3 (3a710): 6.0 mg; Vitamin 1 (3a821): 4.0 mg; Vitamin B2: 16.0 mg; Vitamin B6 (3a831): 4.0 mg; Vitamin B12: 72.0 μg; Nicotinic acid (3a314): 80.0 mg; Pantothenic acid (3a841): 28.0 mg; Choline chloride (3a890): 960.0 mg; Cu (CuSO₄·5H₂O): 40.0 mg; Fe (FeSO₄·H₂O 3b103): 240.0 mg; I (IK 3b201): 12.0 mg; Mn (MnSO₄·H₂O): 560.0 mg; Se (Na₂SeO₃): 0.4 mg; Zn (ZnO 3b603): 0.4 g. In addition, said basal diet may comprise a phytase, e.g. HiPhos 10000, e.g. at 0.006%. Further in addition, said basal diet may comprise one or more of the following: organic acid(s), probiotic(s), zootechnical growth promoter(s) or coccidiostat(s).

In an embodiment, the oligosaccharide preparation of the use described herein is comprised in a nutritional composition administered to the layer bird. In particular embodiments, the oligosaccharide preparation of the use described herein is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed).

In one embodiment, the layer bird whose performance is improved—as described herein is layer poultry (e.g. chicken, duck, goose, turkey, guinea fowl, pigeon, quail), preferably layer chicken.

In a further embodiment, n of the oligosaccharide preparation of the use described herein is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In one embodiment, at least one fraction of the oligosaccharide preparation of the use described herein comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance

In one embodiment, the oligosaccharide preparation of the use described herein has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

In an embodiment, the relative abundance of oligosaccharides in each of the n fractions of the oligosaccharide preparation of the use described herein decreases monotonically with its degree of polymerization.

Merely for clarification, the term “feed conversion per kg eggs” is the ratio of the mass of feed intake by an animal over the mass of eggs produced by said animal. Analogously, the term “feed conversion per dozen eggs” is the ratio of the mass of feed intake by an animal over a dozen eggs produced by said animal. Thus, efficient conversion of food/feed into eggs or egg mass results in a lower feed conversion value, than a less efficient conversion. Consequently, a low feed conversion value is desired.

It is considered that the oligosaccharide preparation may be provided in the form of a powderous formulation comprising at least 20% (w/w) (e.g. at least 20, 25, 30, 35, 40, 45 etc. % w/w) of the oligosaccharide preparation as referred to herein; at least 25% (wt/wt) of a silica-based adsorbate (e.g. diatomaceous earth, amorphous precipitated silica) having an average particle size D of less than or equal to 3000 μm (e.g. 100-500, 200-500, 200-300 μm); and optionally 0-25% (wt/wt) of water and/or an auxiliary substance; wherein the % are based on the total weight of the powderous formulation. For instance, such a powderous formulation may comprise 30-70% (wt/wt) of the oligosaccharide preparation as referred to herein; 30-70% (wt/wt) of a silica based adsorbate (e.g. having an average particle size of at least 50 μm); and 0-21% (wt/wt) of water; wherein the % are based on the total weight of the powderous formulation. In some embodiments the oligosaccharide preparation is formulated as described in any one of Examples 22-26 and 33 of WO 2020/097458.

Descriptions and methods of manufacturing oligosaccharide preparations according to the invention are described in WO 2020/097458 and WO 2016/007778, in particular in the Examples described therein, in particular in any one of Examples 1-7, 16-18 of WO 2020/097458 A1, in the methods described in paragraph [317], and/or in any one of Examples 73-77, 80-89, 97-99, 101-110 of WO 2016/007778 A1. Oligosaccharide preparations according to the invention are characterized by the step of heating an aqueous composition comprising one or more feed sugars and a catalyst to a temperature and for a time sufficient to induce polymerization. Such oligosaccharide preparations may be termed synthetic oligosaccharide preparations. In the following, oligosaccharide preparations as referred to herein are further specified.

Merely for the sake of clarity, the term “oligosaccharide preparation” may refer to a preparation that comprises one or more oligosaccharides.

As used herein, an “oligosaccharide” or “oligomer” may refer to a monosaccharide or a compound containing two or more monosaccharide subunits linked by glycosidic bonds. An “oligosaccharide” may also refer to an anhydro-monosaccharide or a compound containing two or more monosaccharide subunits, where at least one monosaccharide unit is replaced by an anhydro-subunit. An “oligosaccharide” may be optionally functionalized. As used herein, the term “oligosaccharide” encompasses all species of the oligosaccharide, wherein each of the monosaccharide subunit in the oligosaccharide is independently and optionally functionalized and/or replaced with its corresponding anhydro-monosaccharide subunit.

An “anhydro-subunit” may be a product of reversible thermal dehydration of a monosaccharide (or monosaccharide subunit) or a sugar caramelization product. For example, an “anhydro-subunit” may be an anhydro-monosaccharide such as anhydro-glucose. As another example, an “anhydro-subunit” may be linked with one or more regular or anhydro-monosaccharide subunits via glycosidic linkage.

An oligosaccharide may be characterized to contain two or more monosaccharide subunits linked by glycosidic bonds. In this regard, a “gluco-oligosaccharide” may refer to a glucose or a compound containing two or more glucose monosaccharide subunits linked by glycosidic bonds. A “gluco-oligosaccharide” may also refer to an anhydro-glucose or a compound containing two or more glucose monosaccharide subunits linked by glycosidic bonds, wherein at least one monosaccharide subunit is replaced with an anhydro-glucose subunit. Similarly, a “galacto-oligosaccharide” may refer to a galactose or a compound containing two or more galactose monosaccharide subunits linked by glycosidic bonds. A “galacto-oligosaccharide” may also refer to an anhydro-galactose or a compound containing two or more galactose monosaccharide subunits linked by glycosidic bonds, wherein at least one monosaccharide subunit is replaced with an anhydro-galactose subunit. Analogously, a gluco-galactose-oligosaccharide may be a gluco-oligosaccharide, a galacto-oligosaccharide, or a compound containing one or more glucose monosaccharide subunits and one or more galactose monosaccharide subunits linked by glycosidic bonds, wherein at least one of the monosaccharide subunits is replaced with its respective anhydro-monosaccharide subunit. A gluco-galacto-xylo-oligosaccharide may refer to a compound produced by the condensation reaction of glucose, galactose, and xylose. An oligosaccharide preparation comprising gluco-galacto-xylo-oligosaccharides may comprise gluco-galactose-oligosaccharides, gluco-xylo-oligosaccharides, galacto-xylo-oligosaccharides, and compounds containing one or more glucose monosaccharide subunits, one or more xylose monosaccharide subunits, and one or more galactose monosaccharide subunits linked by glycosidic bonds.

As used herein, the term “monosaccharide unit” and “monosaccharide subunit” may be used interchangeably, unless suggested otherwise. A “monosaccharide subunit” may refer to a monosaccharide monomer in an oligosaccharide. For an oligosaccharide having a degree of polymerization of 1, the oligosaccharide may be referred to as a monosaccharide subunit or monosaccharide. For an oligosaccharide having a degree of polymerization higher than 1, its monosaccharide subunits are linked via glycosidic bonds.

As used herein, the term “regular monosaccharide” may refer to a monosaccharide that does not contain an anhydro-subunit. The term “regular disaccharide” may refer to a disaccharide that does not contain an anhydro-subunit. Accordingly, the term “regular subunit” may refer to a subunit that is not an anhydro-subunit.

The term “relative abundance” or “abundance,” as used herein, may refer to the abundance of a species in terms of how common or rare the species exists. For example, a DP1 fraction comprising 10% anhydro-subunit containing oligosaccharides by relative abundance may refer to a plurality of DP1 oligosaccharides, wherein 10%, by number, of the DP1 oligosaccharides are anhydro-monosaccharides.

Degree of Polymerization (DP) Distribution: A distribution of the degree of polymerization of the oligosaccharide preparation may be determined by any suitable analytical method and instrumentation, including but not limited to end group method, osmotic pressure (osmometry), ultracentrifugation, viscosity measurements, light scattering method, size exclusion chromatography (SEC), SEC-MALLS, field flow fractionation (FFF), asymmetric flow field flow fractionation (A4F), high-performance liquid chromatography (HPLC), and mass spectrometry (MS). For example, the distribution of the degree of polymerization may be determined and/or detected by mass spectrometry, such as MALDI-MS, LC-MS, or GC-MS. For another example, the distribution of the degree of polymerization may be determined and/or detected by SEC, such as gel permeation chromatography (GPC). As yet another example, the distribution of the degree of polymerization may be determined and/or detected by HPLC, FFF, or A4F. In another example, the degree of polymerization of the oligosaccharide preparation may be determined based on its molecular weight and molecular weight distribution (for a more detailed description see WO 2020/097458).

Anhydro-subunit Level: In some embodiments, each of the n fractions of oligosaccharides of the oligosaccharide preparation as described herein independently comprises an anhydro-subunit level. For instance, in some embodiments, the DP1 fraction comprises 10% anhydro-subunit containing oligosaccharides by relative abundance, and the DP2 fraction comprises 15% anhydro-subunit containing oligosaccharides by relative abundance. For another example, in some embodiments, DP1, DP2, and DP3 fraction each comprises 5%, 10%, and 2% anhydro-subunit containing oligosaccharides by relative abundance, respectively. In other embodiments, two or more fractions of oligosaccharides may comprise similar level of anhydro-subunit containing oligosaccharides. For example, in some embodiments, the DP1 and DP3 fraction each comprises about 5% anhydro-subunit containing oligosaccharides by relative abundance.

The level of anhydro-subunits may be determined by any suitable analytical methods, such as nuclear magnetic resonance (NMR) spectroscopy, mass spectrometry, HPLC, FFF, A4F, or any combination thereof. In some embodiments, the level of anhydro-subunits is determined, at least in part, by mass spectrometry such as MALDI-MS. In some embodiments, the level of anhydro-subunits may be determined, at least in part, by NMR. In some embodiments, the level of anhydro-subunits may be determined, at least in part, by HPLC. For example, in some embodiments, the level of anhydro-subunits may be determined by MALDI-MS, as illustrated in more detail in WO 2020/097458.

Glycosidic Linkages: In some embodiments, the oligosaccharide preparation described herein comprise a variety of glycosidic linkages. The type and distribution of the glycosidic linkages may depend on the source and manufacturing method of the oligosaccharide preparation. In some embodiments, the type and distribution of various glycosidic linkages may be determined and/or detected by any suitable methods known in the art such as NMR. For example, in some embodiments, the glycosidic linkages are determined and/or detected by proton NMR, carbon NMR, 2D NMR such as 2D JRES, HSQC, HMBC, DOSY, COSY, ECOSY, TOCSY, NOESY, or ROESY, or any combination thereof. In some embodiments, the glycosidic linkages are determined and/or detected, at least in part, by proton NMR. In some embodiments, the glycosidic linkages are determined and/or detected, at least in part, by carbon NMR. In some embodiments, the glycosidic linkages are determined and/or detected, at least in part, by 2D HSQC NMR.

In some embodiments, an oligosaccharide preparation may comprise one or more α-(1,2) glycosidic linkages, α-(1,3) glycosidic linkages, α-(1,4) glycosidic linkages, α-(1,6) glycosidic linkages, β-(1,2) glycosidic linkages, β-(1,3) glycosidic linkages, β-(1,4) glycosidic linkages, β-(1,6) glycosidic linkages, α(1,1)α glycosidic linkages, α(1,1)β glycosidic linkages, β(0,0)β glycosidic linkages, or any combination thereof.

In some embodiments, the oligosaccharide preparations have a glycosidic bond type distribution of about from 0 to 60 mol %, 5 to 55 mol %, 5 to 50 mol %, 5 to 45 mol %, 5 to 40 mol %, 5 to 35 mol %, 5 to 30 mol %, 5 to 25 mol %, 10 to 60 mol %, 10 to 55 mol %, 10 to 50 mol %, 10 to 45 mol %, 10 to 40 mol %, 10 to 35 mol %, 15 to 60 mol %, 15 to 55 mol %, 15 to 50 mol %, 15 to 45 mol %, 15 to 40 mol %, 15 to 35 mol %, 20 to 60 mol %, 20 to 55 mol %, 20 to 50 mol %, 20 to 45 mol %, 20 to 40 mol %, 20 to 35 mol %, 25 to 60 mol %, 25 to 55 mol %, 25 to 50 mol %, 25 to 45 mol %, 25 to 40 mol %, or 25 to 35 mol % of α-(1,6) glycosidic linkages.

Molecular Weight: The molecular weight and molecular weight distribution of the oligosaccharide preparation may be determined by any suitable analytical means and instrumentation, such as end group method, osmotic pressure (osmometry), ultracentrifugation, viscosity measurements, light scattering method, SEC, SEC-MALLS, FFF, A4F, HPLC, and mass spectrometry. In some embodiments, the molecular weight and molecular weight distribution are determined by mass spectrometry, such as MALDI-MS, LC-MS, or GC-MS. In some embodiments, the molecular weight and molecular weight distribution are determined by size exclusion chromatography (SEC), such as gel permeation chromatography (GPC). In other embodiments, the molecular weight and molecular weight distribution are determined by HPLC. In some embodiments, the molecular weight and molecular weight distribution are determined by MALDI-MS.

In some embodiments, the weight average molecular weight of the oligosaccharide preparation is about from 100 to 10000 g/mol, 200 to 8000 g/mol, 300 to 5000 g/mol, 500 to 5000 g/mol, 700 to 5000 g/mol, 900 to 5000 g/mol, 1100 to 5000 g/mol, 1300 to 5000 g/mol, 1500 to 5000 g/mol, 1700 to 5000 g/mol, 300 to 4500 g/mol, 500 to 4500 g/mol, 700 to 4500 g/mol, 900 to 4500 g/mol, 1100 to 4500 g/mol, 1300 to 4500 g/mol, 1500 to 4500 g/mol, 1700 to 4500 g/mol, 1900 to 4500 g/mol, 300 to 4000 g/mol, 500 to 4000 g/mol, 700 to 4000 g/mol, 900 to 4000 g/mol, 1100 to 4000 g/mol, 1300 to 4000 g/mol, 1500 to 4000 g/mol, 1700 to 4000 g/mol, 1900 to 4000 g/mol, 300 to 3000 g/mol, 500 to 3000 g/mol, 700 to 3000 g/mol, 900 to 3000 g/mol, 1100 to 3000 g/mol, 1300 to 3000 g/mol, 1500 to 3000 g/mol, 1700 to 3000 g/mol, 1900 to 3000 g/mol, 2100 to 3000 g/mol, 300 to 2500 g/mol, 500 to 2500 g/mol, 700 to 2500 g/mol, 900 to 2500 g/mol, 1100 to 2500 g/mol, 1300 to 2500 g/mol, 1500 to 2500 g/mol, 1700 to 2500 g/mol, 1900 to 2500 g/mol, 2100 to 2500 g/mol, 300 to 1500 g/mol, 500 to 1500 g/mol, 700 to 1500 g/mol, 900 to 1500 g/mol, 1100 to 1500 g/mol, 1300 to 1500 g/mol, 2000-2800 g/mol, 2100-2700 g/mol, 2200-2600 g/mol, 2300-2500 g/mol, or 2320-2420 g/mol. In some embodiments, the weight average molecular weight of the oligosaccharide preparation is about from 2000 to 2800 g/mol, 2100 to 2700 g/mol, 2200 to 2600 g/mol, 2300 to 2500 g/mol, or 2320 to 2420 g/mol.

Types of Oligosaccharides: In some embodiments, the species of oligosaccharides present in an oligosaccharide preparation referred to herein may depend on the type of the one or more feed sugars. For example, in some embodiments, the oligosaccharide preparations comprise a gluco-oligosaccharide when the feed sugars comprise glucose. For example, in some embodiments, the oligosaccharide preparations comprise a galacto-oligosaccharide when the feed sugars comprise galactose. For another example, in some embodiments, the oligosaccharide preparations comprise gluco-galacto-oligosaccharides when the feed sugars comprise galactose and glucose.

In some embodiments, the oligosaccharide preparations comprise one or more species of monosaccharide subunits. In some embodiments, the oligosaccharide preparation may comprise oligosaccharides with 1, 2, 3, 4, 5, 6, 7, 8, 9, 10, 11, 12, 13, 14, 15, 16, 17, 18, 19, 20, or more different species of monosaccharides subunits.

Method of Manufacturing Oligosaccharide Preparations: The Method of manufacturing an oligosaccharide preparation according to the invention is described in detail in WO 2020/097458, comprising heating an aqueous composition comprising one or more feed sugars and a catalyst to a temperature and for a time sufficient to induce polymerization, wherein the catalyst is selected from the group consisting of: (+)-camphor-10-sulfonic acid; 2-pyridinesulfonic acid; 3-pyridinesulfonic acid; 8-hydroxy-5-quinolinesulfonic acid hydrate; α-hydroxy-2-pyridinemethanesulfonic acid; (β)-camphor-10-sulfonic acid; butylphosphonic acid; diphenylphosphinic acid; hexylphosphonic acid; methylphosphonic acid; phenylphosphinic acid; phenylphosphonic acid; tert-butylphosphonic acid; SS)-VAPOL hydrogenphosphate; 6-quinolinesulfonic acid, 3-(1-pyridinio)-1-propanesulfonate; 2-(2-pyridinyl)ethanesulfonic acid; 3-(2-pyridyl)-5,6-diphenyl-1,2,4-triazine-p,p′-disulfonic acid monosodium salt hydrate; 1,1′-binaphthyl-2,2′-diyl-hydrogenphosphate; bis(4-methoxyphenyl)phosphinic acid; phenyl(3,5-xylyl)phosphinic acid; L-cysteic acid monohydrate; poly(styrene sulfonic acid-co-divinylbenzene); lysine; Ethanedisulfonic acid; Ethanesulfonic acid; Isethionic acid; Homocysteic acid; HEPBS (N-(2-Hydroxyethyl)piperazine-N′-(4-butanesulfonic acid)); HEPES (4-(2-hydroxyethyl)-1-piperazineethanesulfonic acid); 2-Hydroxy-3-morpholinopropanesulfonic acid; 2-(N-morpholino)ethanesulfonic acid; Methanesulfonic acid; Methaniazide; Naphthalene-1-sulfonic acid; Naphthalene-2-sulfonic acid; Perfluorobutanesulfonic acid; 6-sulfoquinovose; Triflic acid; 2-aminoethanesulfonic acid; Benzoic acid; Chloroacetic acid; Trifluoroacetic acid; Caproic acid; Enanthic acid; Caprylic acid; Pelargonic acid; Lauric acid; Pamitic acid; Stearic acid; Arachidic acid; Aspartic acid; Glutamic acid; Serine; Threonine; Glutamine; Cysteine; Glycine; Proline; Alanine; Valine; Isoleucine; Leucine; Methionine; Phenylalanine; Tyrosine; Tryptophan.

In some embodiments, the polymerization of the feed sugars is achieved by a step-growth polymerization. In some embodiments, the polymerization of the feed sugars is achieved by polycondensation.

Feed Sugar: The one or more feed sugars used in the methods of manufacturing oligosaccharide preparations described herein may comprise one or more types of sugars. In some embodiments, the one or more feed sugars comprise monosaccharides, disaccharides, trisaccharides, tetrasaccharides, or any mixtures thereof.

In some embodiments, the one or more feed sugars comprise glucose. In some embodiments, the one or more feed sugars comprise glucose and galactose. In some embodiments, the one or more feed sugars comprise glucose, xylose, and galactose. In some embodiments, the one or more feed sugars comprise glucose and mannose. In some embodiments, the one or more feed sugars comprise glucose and fructose. In some embodiments, the one or more feed sugars comprise glucose, fructose, and galactose. In some embodiments, the one or more feed sugars comprise glucose, galactose, and mannose.

As used herein, the singular forms “a,” “and,” and “the” include plural referents unless the context clearly dictates otherwise. Thus, for example, reference to “an agent” includes a plurality of such agents, and reference to “the oligosaccharide” includes reference to one or more oligosaccharides (or to a plurality of oligosaccharides) and equivalents thereof known to those skilled in the art, and so forth.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The invention is further characterized by the following items:

Item 1: Method for improving performance (e.g. increased body weight gain, increased laying index, increased egg mass, increased percentage of XL eggs, reduced feed conversion per kg eggs and/or reduced feed conversion per dozen eggs) of a layer bird (e.g. layer poultry, layer chicken, layer hen), comprising the steps of
a) providing a layer bird;
b) providing a nutritional composition suitable for layer birds comprising an oligosaccharide preparation (e.g. a synthetic oligosaccharide preparation),
wherein the oligosaccharide preparation comprises at least n fractions of oligosaccharides each having a distinct degree of polymerization selected from 1 to n (DP1 to DPn fractions), wherein n is an integer greater than or equal to 2, and
wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; and
c) administering the nutritional composition to the layer bird.

Item 2: The method according to item 1, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

Item 3. The method according to item 1 or 2, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

Item 4. The method according to any one of the preceding items, wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

Item 5. The method according to any one of the preceding items, wherein the relative abundance of oligosaccharides in each of the n fractions of the oligosaccharide preparation decreases monotonically with its degree of polymerization.

Item 6. The method according to any one of the preceding items, wherein the relative abundance of oligosaccharides in at least 5, 10, 20, or 30 DP fractions of the oligosaccharide preparation decreases monotonically with its degree of polymerization.

Item 7. The method according to any one of the preceding items, wherein the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.

Item 8. The method according to any one of the preceding items, wherein each fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.

Item 9. The method according to any one of the preceding items, wherein at least one fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 40%, 50%, 60%, 70%, or 80% anhydro-subunit containing oligosaccharides by relative abundance.

Item 10. The method according to any one of the preceding items, wherein the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%, 30%, 40%, 50%, 60%, 70%, or 80% anhydro-subunit containing oligosaccharides by relative abundance.

Item 11. The method according to any one of the preceding items, wherein each fraction of the oligosaccharide preparation comprises greater than 20%, 21%, 22%, 23%, 24%, or 25% anhydro-subunit containing oligosaccharides by relative abundance.

Item 12. The method according to any one of the preceding items, wherein more than 99%, 95%, 90%, 85%, 80%, 75%, 70%, 65%, 60%, 55%, 50%, 45%, 40%, 35%, or 30% of the anhydro-subunit containing oligosaccharides of the oligosaccharide preparation have only one anhydro-subunit.

Item 13. The method according to any one of the preceding items, wherein the oligosaccharide preparation has a DP1 fraction content from 1 to 40% by relative abundance.

Item 14. The method according to any one of the preceding items, wherein the oligosaccharide preparation has a DP2 fraction content from 1 to 35% by relative abundance.

Item 16. The method according to any one of the preceding items, wherein the oligosaccharide preparation has a DP3 fraction content from 1 to 30% by relative abundance.

Item 17. The method according to any one of the preceding items, wherein the oligosaccharide preparation has a DP4 fraction content from 0.1 to 20% by relative abundance.

Item 18. The method according to any one of the preceding items, wherein the oligosaccharide preparation has a DP5 fraction content from 0.1 to 15% by relative abundance.

Item 19. The method according to any one of the preceding items, wherein the ratio of DP2 fraction to DP1 fraction of the oligosaccharide preparation is 0.02-0.40 by relative abundance.

Item 20. The method according to any one of the preceding items, wherein the ratio of DP3 fraction to DP2 fraction of the oligosaccharide preparation is 0.01-0.30 by relative abundance.

Item 21. The method according to any one of the preceding items, wherein the aggregate content of DP1 and DP2 fractions in the oligosaccharide preparation is less than 50, 30, or 10% by relative abundance.

Item 22. The method according to any one of the preceding items, wherein the oligosaccharide preparation comprises at least 103, 104, 105, 106 or 109 different oligosaccharide species.

Item 23. The method according to any one of the preceding items, wherein two or more independent oligosaccharides of the oligosaccharide preparation comprise different anhydro-subunits.

Item 24. The method according to any one of the preceding items, wherein the oligosaccharide preparation comprises one or more anhydro-subunits that are products of reversible thermal dehydration of monosaccharides.

Item 25. The method according to any one of the preceding items, wherein the oligosaccharide preparation comprises one or more anhydro-glucose, anhydro-galactose, anhydro-mannose, anhydro-allose, anhydro-altrose, anhydro-gulose, anhydro-indose, anhydro-talose, anhydro-fructose, anhydro-ribose, anhydro-arabinose, anhydro-rhamnose, anhydro-lyxose, or anhydro-xylose subunits.

Item 26. The method according to any one of the preceding items, wherein the oligosaccharide preparation comprises one or more anhydro-glucose, anhydro-galactose, anhydro-mannose, or anhydro-fructose subunits.

Item 27. The method according to any one of the preceding items, wherein the oligosaccharide preparation comprises one or more 1,6-anhydro-β-D-glucofuranose or 1,6-anhydro-β-D-glucopyranose subunits. In some embodiments, the oligosaccharide preparation comprises both 1,6-anhydro-β-D-glucofuranose and 1,6-anhydro-β-D-glucopyranose anhydro-subunits.

Item 28. The method according to any one of the preceding items, wherein a ratio of 1,6-anhydro-β-D-glucofuranose to 1,6-anhydro-β-D-glucopyranose is from about 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in the oligosaccharide preparation.

Item 29. The method according to any one of the preceding items, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 within the oligosaccharide preparation.

Item 30. The method according to any one of the preceding items, wherein the ratio of 1,6-anhydro-β-D-glucofuranose to 1,6-anhydro-β-D-glucopyranose is about 2:1 in the oligosaccharide preparation.

Item 31. The method according to any one of the preceding items, wherein the ratio of 1,6-anhydro-β-D-glucofuranose to 1,6-anhydro-β-D-glucopyranose is about from 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in each fraction of the oligosaccharide preparation.

Item 32. The method according to any one of the preceding items, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 in each fraction of the oligosaccharide preparation.

Item 33. The method according to any one of the preceding items, wherein the ratio of 1,6-anhydro-β-D-glucofuranose to 1,6-anhydro-β-D-glucopyranose is about 2:1 in each fraction of the oligosaccharide preparation.

Item 34. The method according to any one of the preceding items, wherein at least 1%, 5%, 10%, 20%, 30%, 40%, 50%, 60%, 70%, 80%, or 90% of anhydro-subunits in the oligosaccharide preparation are selected from a group consisting of 1,6-anhydro-β-D-glucofuranose and 1,6-anhydro-β-D-glucopyranose.

Item 35: The method according to any one of the preceding items, wherein the weight average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.

Item 36: The method according to any one of the preceding items, wherein the number average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1000 g/mol, 400 to 900 g/mol, 400 to 800 g/mol, 500 to 900 g/mol, or 500 to 800 g/mol.

Item 37: The method according to any one of the preceding items, wherein the distribution of the degree of polymerization is determined and/or detected by MALDI-MS, GC-MS, LC-MS, SEC, HPLC and/or combination(s) thereof (e.g. MALDI-MS and SEC).

Item 38: The method according to any one of the preceding items, wherein the degree of polymerization of the oligosaccharide preparation may be determined based on its molecular weight and molecular weight distribution.

Item 39. The method according to any one of the preceding items, wherein the oligosaccharide preparation is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed); and/or wherein the oligosaccharide preparation comprised in the nutritional composition at an inclusion rate of at least 50 ppm (e.g. at least 50, 70, 100, 150, 200, 300, 400, 500 ppm); and/or wherein the oligosaccharide preparation comprised in the nutritional composition at a concentration of at least 50 ppm (e.g. at least 50, 70, 100, 150, 200, 300, 400, 500 ppm).

Item 40. The method according to any one of the preceding items, wherein the performance is improved on at least one day within 22-35 weeks of age of the layer bird, preferably on at least 2 days, more preferably on at least 3 days within 22-35 weeks of age of the layer bird.

Item 41. The method according to any one of the preceding items, wherein the nutritional composition is administered for at least one day, preferably for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109,110, 111, 112, 113, 114, 115, 116, 117, 118, 119, 120, 121, 122, 123, 124, 125, 126, 127, 128, 129, 130, 131,132, 133, 134, 135, 136, 137, 138, 139, 140, 141, 142, 143, 144, 145, 146, 147, 148, 149, 150, 151,152, 153, 154, 155, 156, 157, 158, 159, 160, 161, 162, 163, 164, 165, 166, 167,168, 169, 170, 171, 172, 173, 174, 175, 176, 177, 178, 179, 180, 181, 182, 183, 184, 185,186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221,222, 223, 224, 225, 226, 227, 228, 229, 230, 231, 232, 233, 234, 235, 236, 237, 238, 239, 240, 241, 242, 243, 244, or 245 days, most preferably, the nutritional composition is administered continuously, i.e. uninterruptedly.

Item 42. Egg obtained by the method of any one of the preceding items.

Item 43. Use of an oligosaccharide preparation (e.g. synthetic oligosaccharide preparation) to improve the performance (e.g. increased body weight gain, increased laying index, increased egg mass, increased percentage of XL eggs, reduced feed conversion per kg eggs and/or reduced feed conversion per dozen eggs) of a layer bird (e.g. layer poultry, layer chicken, layer hen),
wherein the oligosaccharide preparation comprises at least n fractions of oligosaccharides each having a distinct degree of polymerization selected from 1 to n (DP1 to DPn fractions), wherein n is an integer greater than or equal to 2; and
wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

Item 44. The use according to item 43, wherein the oligosaccharide preparation is administered to the bird before, after, and/or simultaneously with the diet of the layer bird, preferably simultaneously with the diet.

Item 45. The use according to item 43 or 44, wherein the oligosaccharide preparation is comprised in a nutritional composition administered to the layer bird.

Item 46. The use according to item 45, wherein the oligosaccharide preparation is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed).

Item 47. The use according to any one of items 43-46, wherein the layer bird is layer poultry (e.g. chicken, duck, goose, turkey, guinea fowl, pigeon, quail), preferably layer chicken.

Item 48. The use according to any one of items 43-47, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

Item 49. The use according to any one of items 43-48, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

Item 50. The use according to any one of items 43-49, wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

Item 51. The use according to any one of items 43-50, wherein the relative abundance of oligosaccharides in each of the n fractions of the oligosaccharide preparation decreases monotonically with its degree of polymerization.

Item 52. The use according to any one of items 43-51, wherein the relative abundance of oligosaccharides in at least 5, 10, 20, or 30 DP fractions of the oligosaccharide preparation decreases monotonically with its degree of polymerization.

Item 53. The use according to any one of items 43-52, wherein the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.

Item 54. The use according to any one of items 43-53, wherein each fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.

Item 55. The use according to any one of items 43-54, wherein at least one fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 40%, 50%, 60%, 70%, or 80% anhydro-subunit containing oligosaccharides by relative abundance.

Item 56. The use according to any one of items 43-55, wherein the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%, 30%, 40%, 50%, 60%, 70%, or 80% anhydro-subunit containing oligosaccharides by relative abundance.

Item 57. The use according to any one of items 43-56, wherein each fraction of the oligosaccharide preparation comprises greater than 20%, 21%, 22%, 23%, 24%, or 25% anhydro-subunit containing oligosaccharides by relative abundance.

Item 58. The use according to any one of items 43-57, wherein more than 99%, 95%, 90%, 85%, 80%, 75%, 70%, 65%, 60%, 55%, 50%, 45%, 40%, 35%, or 30% of the anhydro-subunit containing oligosaccharides of the oligosaccharide preparation have only one anhydro-subunit.

Item 59. The use according to any one of items 43-58, wherein the oligosaccharide preparation has a DP1 fraction content from 1 to 40% by relative abundance.

Item 60. The use according to any one of items 43-59, wherein the oligosaccharide preparation has a DP2 fraction content from 1 to 35% by relative abundance.

Item 61. The use according to any one of items 43-60, wherein the oligosaccharide preparation has a DP3 fraction content from 1 to 30% by relative abundance.

Item 62. The use according to any one of items 43-61, wherein the oligosaccharide preparation has a DP4 fraction content from 0.1 to 20% by relative abundance.

Item 63. The use according to any one of items 43-62, wherein the oligosaccharide preparation has a DP5 fraction content from 0.1 to 15% by relative abundance.

Item 64. The use according to any one of items 43-63, wherein the ratio of DP2 fraction to DP1 fraction of the oligosaccharide preparation is 0.02-0.40 by relative abundance.

Item 65. The use according to any one of items 43-64, wherein the ratio of DP3 fraction to DP2 fraction of the oligosaccharide preparation is 0.01-0.30 by relative abundance.

Item 66. The use according to any one of items 43-65, wherein the aggregate content of DP1 and DP2 fractions in the oligosaccharide preparation is less than 50, 30, or 10% by relative abundance.

Item 67. The use according to any one of items 43-66, wherein the oligosaccharide preparation comprises at least 103, 104, 105, 106 or 109 different oligosaccharide species.

Item 68. The use according to any one of items 43-67, wherein two or more independent oligosaccharides of the oligosaccharide preparation comprise different anhydro-subunits.

Item 69. The use according to any one of items 43-68, wherein the oligosaccharide preparation comprises one or more anhydro-subunits that are products of reversible thermal dehydration of monosaccharides.

Item 70. The use according to any one of items 43-69, wherein the oligosaccharide preparation comprises one or more anhydro-glucose, anhydro-galactose, anhydro-mannose, anhydro-allose, anhydro-altrose, anhydro-gulose, anhydro-indose, anhydro-talose, anhydro-fructose, anhydro-ribose, anhydro-arabinose, anhydro-rhamnose, anhydro-lyxose, or anhydro-xylose subunits.

Item 71. The use according to any one of items 43-70, wherein the oligosaccharide preparation comprises one or more anhydro-glucose, anhydro-galactose, anhydro-mannose, or anhydro-fructose subunits.

Item 72. The use according to any one of items 43-71, wherein the oligosaccharide preparation comprises one or more 1,6-anhydro-β-D-glucofuranose or 1,6-anhydro-β-D-glucopyranose subunits. In some embodiments, the oligosaccharide preparation comprises both 1,6-anhydro-β-D-glucofuranose and 1,6-anhydro-β-D-glucopyranose anhydro-subunits.

Item 73. The use according to any one of items 43-72, wherein a ratio of 1,6-anhydro-β-D-glucofuranose to 1,6-anhydro-β-D-glucopyranose is from about 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in the oligosaccharide preparation.

Item 74. The use according to any one of items 43-73, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 within the oligosaccharide preparation.

Item 75. The use according to any one of items 43-74, wherein the ratio of 1,6-anhydro-β-D-glucofuranose to 1,6-anhydro-β-D-glucopyranose is about 2:1 in the oligosaccharide preparation.

Item 76. The use according to any one of items 43-75, wherein the ratio of 1,6-anhydro-β-D-glucofuranose to 1,6-anhydro-β-D-glucopyranose is about from 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in each fraction of the oligosaccharide preparation.

Item 77. The use according to any one of items 43-76, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 in each fraction of the oligosaccharide preparation.

Item 78. The use according to any one of items 43-77, wherein the ratio of 1,6-anhydro-β-D-glucofuranose to 1,6-anhydro-β-D-glucopyranose is about 2:1 in each fraction of the oligosaccharide preparation.

Item 79. The use according to any one of items 43-78, wherein at least 1%, 5%, 10%, 20%, 30%, 40%, 50%, 60%, 70%, 80%, or 90% of anhydro-subunits in the oligosaccharide preparation are selected from a group consisting of 1,6-anhydro-β-D-glucofuranose and 1,6-anhydro-β-D-glucopyranose.

Item 80. The use according to any one of items 43-79, wherein the weight average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.

Item 81. The use according to any one of items 43-80, wherein the number average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1000 g/mol, 400 to 900 g/mol, 400 to 800 g/mol, 500 to 900 g/mol, or 500 to 800 g/mol.

Item 82. The use according to any one of items 43-81, wherein the distribution of the degree of polymerization is determined and/or detected by MALDI-MS, GC-MS, LC-MS, SEC, HPLC and/or combination(s) thereof (e.g. MALDI-MS and SEC).

Item 83. The use according to any one of items 43-82, wherein the degree of polymerization of the oligosaccharide preparation may be determined based on its molecular weight and molecular weight distribution.

Item 84. The use according to any one of items 43-83, wherein the oligosaccharide preparation is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed); and/or wherein the oligosaccharide preparation comprised in the nutritional composition at an inclusion rate of at least 50 ppm (e.g. at least 50, 70, 100, 150, 200, 300, 400, 500 ppm); and/or wherein the oligosaccharide preparation comprised in the nutritional composition at a concentration of at least 50 ppm (e.g. at least 50, 70, 100, 150, 200, 300, 400, 500 ppm).

Item 85. The use according to any one of items 43-84, wherein the performance is improved on at least one day within 22-35 weeks of age of the layer bird, preferably on at least 2 days, more preferably on at least 3 days within 22-35 weeks of age of the layer bird.

Item 86. The use according to any one of items 43-85, wherein the nutritional composition is administered for at least one day, preferably for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113,114, 115, 116, 117, 118, 119,120, 121, 122, 123, 124, 125, 126, 127, 128, 129, 130, 131,132, 133, 134, 135, 136, 137, 138, 139, 140, 141, 142, 143, 144, 145, 146, 147, 148, 149,150, 151, 152, 153, 154, 155, 156, 157, 158, 159,160, 161, 162, 163, 164, 165, 166, 167, 168, 169, 170,171, 172, 173, 174, 175, 176, 177, 178, 179, 180, 181, 182, 183, 184, 185,186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, or 245 days, most preferably, the nutritional composition is administered continuously, i.e. uninterruptedly.

The oligosaccharide preparation referred to herein may be characterized by any one, two or more or even all of the individual features as described in the items above. In other words, the oligosaccharide preparation may be characterized by any combination of the individual features of the oligosaccharide preparation described in the items above. For instance, in a particular embodiment of a method according to the invention, the oligosaccharide preparation may be characterized by a combination of the combined oligosaccharide preparation features as described in items 3, 5, 17 and 25.

In the following, the present invention is further described by non-limiting examples. The present invention as disclosed herein is not limited to specific embodiments, figures, methodology, examples, protocols etc. described herein but solely defined by the claims.

### EXAMPLES

### Example 1—Effect of Oligosaccharide Preparation on Layer Bird Performance

A feeding trial was performed to study the effects of oligosaccharide preparations on the performance of layer birds.

Oligosaccharide preparations were prepared as disclosed in WO 2020/097458 and WO 2016/007778, in particular in any one of Examples 1-7, 16-18 of WO 2020/097458 A1, in the methods described in paragraph [317], and/or in any one of Examples 73-77, 80-89, 97-99, 101-110 of WO 2016/007778 A1. The oligosaccharide preparations comprise at least n fractions of oligosaccharides each having a distinct degree of polymerization selected from 1 to n (DP1 to DPn fractions), wherein n is an integer greater than 3; wherein each of a DP1 and DP2 fraction independently comprises from about 0.5% to about 15% of anhydro-subunit containing oligosaccharides by relative abundance as determined by mass spectrometry.

Experimental animals: Birds arrived at the experimental facilities with ca. 18 weeks of age and adaptation period (nutrition and photoperiod) was applied. A total of 210 22-weeks old Isa Brown laying hens were included in the study and allocated to the experimental treatments. Birds were of average weight as per the ISA Brown standards at 22 weeks of age (mean 1,695 g; standard deviation 73.7 g). The breeder flock and vaccination history were recorded. Prior to the start of the study, the animals were examined for signs of ill, health or injury. Any layer that appears to be in poor condition was removed from the study. During the experimental period, each animal was observed daily by the animal supervisor in its cage and any variation of its appearance, the appearance of its excreta or its behaviour was noted. Any bird judged unlikely to survive or to be suffering pain or distress was humanely euthanized and most probable cause of poor condition was noted. Culled and dead birds were weighed, and date recorded.

Experimental design: A Randomized Complete Block Design (RCBD) with 2 experimental treatments was used: “T1” was basal feed as control. “T2” was the same basal feed further comprising 900 g of an oligosaccharide preparation as described herein per ton of feed. Treatments were balanced by initial weight and laying index at the onset of the study.

Randomization: Each treatment was replicated 21 times and 5 hens housed together formed the experimental unit. The total space was divided into 7 blocks to account for any environmental variation. Treatments were then assigned at random to the pens within the blocks (3 pens per treatment in each block). A random sequence was generated for 4 pens of each block (Table 9:2). Every sequence was repeated within the respective blocks (Figure 9:1) in order to ensure a uniform distribution across the house.

Method for reducing bias: Animals and cages were randomised to treatments, which were blinded to the barn crew with coded colours and numerical codes. Additionally, colour-coded feed bags and cages matched the colour codes for treatments.

Experimental diets: Layers received a basal diet for the first 3-4 weeks. This diet was the same as used for the present study. The experimental diets were calculated to be isonutritive, and to meet or exceed the nutrient requirements recommended by NRC (1994) for layers. The composition of feed ingredients is shown in Table 1 below. The diets (mash) and water were ad libitum available.

Feeds were prepared before the trial start to allow time for checking the experimental product content and homogeneity. Oligosaccharide preparation was premixed with ground cereal before addition to the final mix to assure fine homogeneity. Diets were prepared without the inclusion of any enzymes (except for a phytase, HiPhos 10 000), organic acids, probiotics, zootechnical growth promoters or coccidiostat (Table 1) and they were analysed for nutritional homogeneity (Table 2). In order to avoid cross-contamination, T1 feed was manufactured before T2 feed. All diets were stored in a cool (<20° C.) and dry place until used.

TABLE 1
Composition and calculated analyses of the experimental diet.

| | Basal diet |
|---|---|
| Ingredients, % | |
| Maize | 31.730 |
| Wheat | 25.000 |
| Sunflower meal | 10.000 |
| Soybean meal | 18.972 |
| Soy oil | 3.989 |
| Calcium carbonate fine | 2.583 |
| Calcium carbonate coarse | 6.026 |
| Monocalcium phosphate | 0.549 |
| Salt | 0.356 |
| DL-Methionine | 0.193 |
| L-Lysine HCl | 0.161 |
| L-Threonine | 0.029 |
| L-Tryptophan | 0.007 |
| Vit&Min premix* | 0.400 |
| HiPhos 10000 | 0.006 |
| Calculated analysis, % | |
| Dry Matter | 89.24 |
| Ash | 11.83 |
| Crude Protein | 17.00 |
| Ether Extract | 5.87 |
| Crude Fibre | 4.48 |
| Neutral Detergent Fibre | 11.18 |
| Starch | 35.57 |
| Calcium | 3.800 |
| Total Phosphorus | 0.507 |
| Phytic P | 0.280 |
| Av. Phosphorus | 0.380 |
| Sodium | 0.160 |
| Chloride | 0.296 |
| AMEn, kcal/kg | 2767 |
| AMEn, MJ/kg | 11.58 |
| Total Lysine | 0.91 |
| Total Methionine | 0.47 |
| Total Met + Cys | 0.77 |
| Total Threonine | 0.64 |
| Total Tryptophan | 0.21 |
| Dig. Lysine | 0.800 |
| Dig. Methionine | 0.446 |
| Dig. Met + Cys | 0.680 |
| Dig. Threonine | 0.540 |
| Dig. Tryptophan | 0.180 |
| Dig. Isoleucine | 0.605 |
| Dig. Valine | 0.691 |
| Dig. Arginine | 1.011 |

*Provided per kilogram of diet: Vitamin A (3a672a): 40,000 IU; Vitamin D3 (3a671): 8,000 IU; Vitamin E (α-tocopherol 3a700): 40.0 mg; Vitamin K3 (3a710): 6.0 mg; Vitamin B1 (3a821): 4.0 mg; Vitamin B2: 16.0 mg; Vitamin B6 (3a831): 4.0 mg; Vitamin B12: 72.0 μg; Nicotinic acid (3a314): 80.0 mg; Pantothenic acid (3a841): 28.0 mg; Choline chloride (3a890): 960.0 mg; Cu (CuSO₄•5H₂O): 40.0 mg; Fe (FeSO₄•H₂O 3b103): 240.0 mg; I (IK 3b201): 12.0 mg; Mn (MnSO₄•H₂O): 560.0 mg; Se (Na₂SeO₃): 0.4 mg; Zn (ZnO 3b603): 0.4 g.

TABLE 2
Analytical results of experimental diets.

| Nutrient, % | Method of analysis | T1 | T2 |
|---|---|---|---|
| Dry Matter | Oven drying | 10.20 | 10.10 |
| Crude Protein | Kjeldahl | 16.80 | 16.90 |
| Crude Fibre | Weende | 4.70 | 4.50 |
| Ether Extract | Soxhlet | 5.90 | 5.60 |
| Ash | Incineration | 12.34 | 12.21 |
| Calcium | Atomic absorption spectroscopy | 3.75 | 3.64 |
| Phosphorous | UV-vis spectroscopy | 0.47 | 0.43 |
| Starch | Polarimetric | 36.40 | 35.70 |

Feed sampling plan: Samples of all feeds were grabbed immediately before bagging at intervals of 40 kg feed. These grab samples were pooled to form a composite and representative sample of each batch of diets. A total of 3,300 g composite sample from each treatment were divided in different samples in sealed plastic bags and sent for nutrient analysis.

Experimental facilities and housing: The study site is detailed in section 3 Study contacts. The laying hens were kept in groups of 5 per cage (0.153 m2/hen) in a clean house equipped with 84 cages. All cages were equipped with perforated plate claw shorteners, perches, a litter area and one nest box. The building was supplied with artificial, programmable lights, automated electric heating and forced ventilation. Temperature and lighting program were adjusted according to the age and productive stage of the animals.

Veterinary care and concomitant treatment: The study animals were in good health and nutritional status before initiation of the study as determined by a veterinary surgeon. Neither collective nor individual veterinary treatments were administered.

Experimental procedures: All records were dated:
Body weight of the layers at the beginning (1 week before the onset of the study), and at the end of the study.
Zootechnical performance parameters. Egg production (number of laid eggs) was recorded daily. Feed intake (g/day) and egg mass were recorded or calculated for the whole experiment. Egg production (number of eggs produced per day and 100 birds in “% lay”), feed intake (g/day), egg mass and feed efficiency (g feed/g egg and kg feed/dozen eggs) were calculated from the collected data for the whole study.
Egg weight and classification. At 4, 8, 12, 16 and 20 weeks on trial, all eggs laid in a 24-hour period were collected, weighed individually and classified commercially (S, M, L, XL), where “S” indicates a weight of less than 52.5 g; “M” indicates a weight of equal to or more than 52.5 g, but less than 62.5 g; “L” indicates a weight of equal to or more than 62.5 g, but less than 72.5 g; and “XL” indicates a weight of more than 72.5 g.
General health records (e.g. faeces consistency, respiratory problems, leg problems etc.). Most likely causes of culling, illness and mortality were recorded. Mortality and weight of dead birds were recorded as it occurs and percentage mortality calculated.
No unusual incidents/adverse events happened (abrupt changes in the weather, disease outbreaks, power failures, feed/water blockages, frozen/burst pipes, etc.)

Statistical analysis—experimental unit: The basic study design was a RCBD, and the cage was the experimental unit. Performance parameters were determined as mean pen values.

Statistical analysis—Sample size justification: Power calculations were done assuming a bilateral test with a Type I error of 5% (α=0.05) and a Type II error of 20% (power 1−β=0.8) and estimated that 21 cages per treatment group were enough to detect a Least Significant Difference (LSD) of 1.43 units (1.5%) between groups for the pivot variable Laying rate (Table 3) for 22-34 weeks of age.

TABLE 3
Power analysis.
t tests - Means: Difference between two independent means (two groups)

| | | Sensitivity: Compute |
|---|---|---|
| Analysis: | | required effect size |
| Input: | Tail(s) | Two |
| | α err prob | 0.05 |
| | Power (1-β err prob) | 0.8 |
| | Sample size group 1 | 21 |
| | Sample size group 2 | 21 |
| Output: | Noncentrality parameter δ | 2.8711284 |
| | Critical t | 2.0210754 |
| | Df | 40 |
| | Effect size d | 0.8860494 |

Theoretical data used to estimate the Least Significant
Difference (LSD) with the calculated d

| | Control group | | Experimental group | | |
|---|---|---|---|---|---|
| | (historical data) | | Expected | | \|LSD\| |
| Parameter | Mean | SD | mean | \|LSD\| | % |
| Laying rate 22- | 97.189 | 1.6093 | 98.61 | 1.43 | 1.5 |
| 34 wks % | | | | | |
| FCR 22-34 wks | 1.9697 | 0.0957 | 1.885 | 0.085 | 4.3% |
| kg feed/kg egg | | | | | |

Statistical analysis—Significance level: Statistical significance was declared at P≤0.05, with 0.05<P≤0.10 considered as a near-significant trend.

Statistical analysis—Statistical methods: To identify the outliers, the interquartile range (iqr) was used. When one value was higher than 3 iqr for any of the zootechnical performance data (laying index, egg weight, egg mass, average daily feed intake (ADFI), feed conversion ratio (FCR)) in two or more of the parameters measured, the pen was considered as outlier for the whole trial. There was no missing data.

The statistical analysis was performed with SPSS (v. 27.0).

Productive parameters were analysed using a General Linear Model (GLM) once the test conditions were checked. The model considered was as follows:

$${Y}_{ijk} = μ + {TREATMENT}_{i} + {BLOCK}_{j} + {e}_{ijk}$$

Where Yᵢⱼₖ is the analyzed variable of the cage k, of the treatment group i (TREATMENT), starting day j (BLOCK), and being the experimental error, eᵢⱼₖ. The results are presented by periods in tables by using least squares corrected means and its standard error. Means are separated with Tukey post-hoc comparison test.

Results: Zootechnical performance and general health of the laying hens were good with no disease problems, and no antibiotic use. The effect of treatment on the performance of laying hens is summarized in Table 4. Supplementation of the feed with an oligosaccharide preparation as described herein increased the body weight (BW) of hens at the end of trial and BW gain when compared to the Controls. During the evaluated time period (22-35 weeks of age), supplementation with the oligosaccharide preparation significantly increased laying index and egg mass, reduced feed conversion per kg and per dozen eggs, and increased the percentage of XL eggs, compared to the control group. In conclusion, supplementation with the oligosaccharide preparation significantly improved performance of layer birds by means of increased body weight gain, increased laying index, increased egg mass and increased percentage of XL eggs, and reduced feed conversion per kg and per dozen eggs.

TABLE 4
Performance of layer birds.

| | | | SEM | P |
|---|---|---|---|---|
| Parameter | T1 | T2 | (n = 21) | (probability) |
| Global trial period (0-140 d) | | | | |
| Initial BW, g | 1691 | 1689 | 15.6 | 0.9252 |
| Final BW, g | 1825^{b} | 1879^{a} | 18.8 | 0.0485 |
| BW gain, g | 133^{b} | 190^{a} | 17.3 | 0.0270 |
| Laying index, % | 90.9^{b} | 95.0^{a} | 0.94 | 0.0038 |
| Egg weight, g | 60.6 | 61.5 | 0.40 | 0.1095 |
| Egg mass, g/hen/d | 55.0^{b} | 58.4^{a} | 0.62 | 0.0004 |
| ADFI, g/d | 124.5 | 125.6 | 1.49 | 0.6123 |
| FC per kg egg | 2.27^{b} | 2.15^{a} | 0.027 | 0.0047 |
| FC per dozen eggs | 1.65^{b} | 1.59^{a} | 0.019 | 0.0311 |
| Mortality, % | 0.9 | 0.0 | 0.68 | 0.3475 |
| Commercial classification, % | | | | |
| S eggs | 1.5 | 1.7 | 0.72 | 0.8452 |
| M eggs | 69.0 | 61.3 | 3.99 | 0.1839 |
| L eggs | 29.3 | 33.2 | 4.03 | 0.4948 |
| XL eggs | 0.2^{b} | 3.7^{a} | 1.04 | 0.0225 |

SEM: standard error of the mean (n: number of observations).
Different superscript letters in the same row show statistical differences (^{a, b}P ≤ 0.05).

### Example 2—Effect of Oligosaccharide Preparation on Layer Birds Challenged with Salmonella

In order to study the effects of an oligosaccharide preparation according to the present invention on layer birds being challenged with Salmonella, a feeding trial was conducted.

Animals and housing: Seventy-two Hy-Line® W-36 layer hens at the early production phase were used. Two birds were placed in each wire-cage and fed a corn-soy layer mash diet. One room was utilized during this experiment. Total of 36 cages was utilized from the room. This room had a 16 h light and 8 h dark photoperiod and temperature was controlled at 24±1° C. Eggs were collected and recorded every day at 3:30 PM. Hens used in this study were not previously vaccinated with Salmonella. Prior to trial start, facilities as well as birds were screened for previous Salmonella enteritidis infection.

Feed and feeding: Individual feeders were placed in each experimental unit, feed was added every day. Feed was formulated as per the Hy-Line® management guide for that phase. Corn-soy based mash diet were provided. The amount of feed consumed was recorded every day in the afternoon to calculate the feed intake. Feed and water was provided ad libitum.

Adaptation period: A one-week adaption period was given for the birds in the trial facility before the experiment. During the adaptation period the birds were given ad libitum feed and water as per the phase of the birds. Birds were fed the experimental diet throughout the adaptation period and the trial.

Challenge protocol: Layer birds were challenged with nalidixic acid resistant Salmonella enteritidis (SENAR). To prepare the inoculum, single SENAR colonies were transferred into XLT4 Agar (agar, 18 g/L; ammonium-iron(III) citrate, 0.8 g/L; lactose, 7.5 g/L; L-lysine, 5 g/L; phenol-red, 0.08 g/L; proteose peptone No. 3, 1.6 g/L; sodium chloride, 5 g/L; sodium thiosulfate, 6.8 g/L; sucrose, 7.5 g/L; xylose, 3.75 g/L; yeast extract, 3 g/L) containing 200 ppm of nalidixic acid, and were incubated at 37° C. for 24 h. The isolated colonies were transferred to 3 mL of tryptic soy broth (TSB) (ThermoFisher Scientific™, San Jose, CA) and incubated at 37° C. for 12 h. The absorbance value for 10⁸ colony-forming units (cfu) per mL was determined and bacterial concentration was confirmed by streaking into a XLT4 plate and enumerating the colonies with corresponding values with the spectrophotometer reading of the 2-fold dilution. For incoulation, feed withdrawal was performed 12 hours prior to SENAR challenge. Each of the birds was challenged individually with 1 mL of inoculum. The birds were inoculated through the oral route using a tuberculin syringe (BD Difco™, Franklin Lakes, NJ) on 0 and 1 d of the experiment. The inoculum had 10⁸ cfu/mL of SENAR as elaborated in Table 5. The test product used in treatments T1 and T2 was an oligosaccharide preparation according to the present invention. In particular the oligosaccharide preparations were prepared as disclosed in WO 2020/097458 and WO 2016/007778, in particular in any one of Examples 1-7, 16-18 of WO 2020/097458 A1, in the methods described in paragraph [317], and/or in any one of Examples 73-77, 80-89, 97-99, 101-110 of WO 2016/007778 A1. The oligosaccharide preparations comprise at least n fractions of oligosaccharides each having a distinct degree of polymerization selected from 1 to n (DP1 to DPn fractions), wherein n is an integer greater than 3; wherein each of a DP1 and DP2 fraction independently comprises from about 0.5% to about 15% of anhydro-subunit containing oligosaccharides by relative abundance as determined by mass spectrometry.

TABLE 5
Salmonella challenge trial overview.

| | Test product | SE Challenge | Number | Number |
|---|---|---|---|---|
| Treatment | (inclusion rate) | (2 × 10⁸ CFU) | of cages | of birds |
| Negative Control | No | No | 9 | 18 |
| (NC) | | | | |
| Positive Control | 0 | Yes | 9 | 18 |
| (PC) | | | | |
| T1 | 450 | Yes | 9 | 18 |
| T2 | 900 | Yes | 9 | 18 |

Sample collection and processing—Feces: Fecal shedding was collected on 0, 3, 7, 10 days post-inoculation (dpi). A metal tray was placed under each cage to collect the feces. Disposable latex gloves were used to collect samples and changed between experimental units. Approximately 20 g of sample was collected using a disposable tongue depressor in a sterile whirl Pak bag. Excessive fecal material in the tray was scraped off using a metal scraper. 200 mL of pre-enrichment (Buffer Peptone Water; BPW) was added to each sample in a ratio of 1:10 (sample to BPW) and mixed thoroughly using sterile plastic loops. 1 mL of pre-enriched sample was transferred for enrichment in 10 mL of tetrathionate enrichment broth and 0.1 mL of samples was inoculated in 9.9 mL of Modified Semisolid Rappaport Vassiliadis (MSRV) broth and incubated at 42° C. for 24 h. The samples were serially diluted in Phosphate Buffer Saline (PBS). A 100 μL sample was further inoculated in XLT4 plate with 200 ppm nalidixic acid using spread plate technique with 5 serial dilutions and incubated at 37° C. for 24 h. Black centered colonies were counted to enumerate the concentration of SENAR.

Sample collection and processing—Tissue: One hen from each cage was euthanized using carbon dioxide at 7 and 14 dpi. The birds were weighed and internal organs like liver with gall bladder, cecal contents, and ovaries were collected, weighed, and transferred aseptically in a sterile plastic bag. A 10 g sample from the liver with gall bladder and ovaries was triturated using a rubber mallet and transferred to tetrathionate enrichment broth in a ratio of 1:10. Samples were incubated at 37° C. for 24 h. 100 μL of the enriched sample was further inoculated in XLT4 agar plate, using the spread plate technique with 5 serial dilutions and colonies were counted after incubation at 37° C. for 24 h.

Sample collection and processing—Serology: Serum was collected on 0, 7, and 14 dpi and analyzed for the presence of Salmonella antibodies using BioChek Salmonella ELISA kit (BioChek) according to the manufactures' protocol.

Sample collection and processing—Microbiota: Approximately 150 mg of cecal content from each sample was used to extract DNA using Quick-DNA™ Fecal/Soil Microbe Kits (Zymo Research, USA) according to the manufacturer's instructions. V4 region of 16S rRNA gene from genomic DNA of each sample was amplified using forward 515F and reverse 806R primers. PCR was conducted using Platinum™ II Hot-Start Green PCR Master Mix (2×) (Thermo fisher Scientific, Catalog No. 14000013). The thermocycling condition of PCR was an initial denaturation step at 94° C. for 2 min, 35 cycles of 0.5 min at 94° C., 0.5 min at 60° C., and 0.5 min at 68° C., and a final extension of 5 min at 68° C. The length of amplicons was confirmed by running on 1% Agarose gel electrophoresis. 300 ng of each sample was measured using Qubit dsDNA BR Assay Kit (Thermo Fisher Scientific) and pooled together. The pooled samples were loaded on 1% agarose gel electrophoresis and purified using Zymoclean Gel DNA Recovery Kit (Zymo Research, Catalog No. D4007). Purified amplicons were further sequenced with Illumina MiSeq paired end 300 cycle options.

Sample collection and processing—Statistical analysis: The data was analyzed using Proc GLM procedure of SAS version 9.4 (SAS Inc., Cary, NC). Fecal Salmonella concentration was log-transformed and analyzed as a completely randomized design with a split-plot in time (0, 3, and 7 dpi). Tissue Salmonella concentration at 7 dpi was log-transformed and analyzed as an CRD. The Means were separated using Fisher's protected LSD, and differences were considered significant at P≤0.05.

Results: Upon feeding of the oligosaccharide preparations in T1 and T2, reduction in Salmonella counts was found. Most notably, significantly lower fecal shedding was observed for T1 and T2 as compared to PC at 7 dpi, see Table 6. As a consequence, products such as eggs obtained from layers having received an oligosaccharide preparation according to the present invention have a significantly lower risk of Salmonella contamination and comprise fewer Salmonella cfu than products obtained from layers that did not receive an oligosaccharide preparation according to the present invention. In addition and analogous to the results shown in Example 1, egg productivity was found improved upon providing the oligosaccharide preparations in T1 and T2 as compared to PC. For instance, the HDEP (hen-day egg production) values at day 14 were 100, 87, 92 and 89 at feed intake (FI) values of 120, 117, 110 and 103 for the treatments NC, PC, T1 and T2, respectively. In Table 7, average body weights and organ weights are summarized.

TABLE 6
log cfu of Salmonella per gram count from various
organs and fecal samples at 0, 7 and 14 dpi.

| Day | Organ | PC | T1 | T2 | SEM | P-value |
|---|---|---|---|---|---|---|
| 0 | Fecal | 0 | 0 | 0 | | |
| 7 | Fecal | 0.55^{a} | 0.21^{b} | 0.18^{b} | 0.1142 | 0.0420 |
| 7 | Liver | 0.44 | 0 | 0 | 0.174 | 0.1301 |
| 7 | Ceca | 0.77 | 0.29 | 0 | 0.2353 | 0.0729 |
| 14 | Fecal | 0.055 | 0 | 0 | 0.0220 | 0.1247 |

TABLE 7
Average body weight, relative liver, spleen,
and ovary weight in layer birds.

| | | Body | | | |
|---|---|---|---|---|---|
| Treatment | Day | weight (g) | Liver (%) | Spleen (%) | Ovary (%) |
| NC | 7 | 1676 | 3.74^{a} | 0.082 | 2.62 |
| PC | 7 | 1659 | 3.86^{a} | 0.089 | 2.75 |
| T1 | 7 | 1665 | 3.90^{a} | 0.091 | 2.66 |
| T2 | 7 | 1744 | 3.54^{ab} | 0.087 | 2.48 |
| NC | 14 | 1660 | 3.24^{bc} | 0.09 | 3 |
| PC | 14 | 1558 | 3.09^{c} | 0.107 | 2.49 |
| T1 | 14 | 1612 | 3.63^{b} | 0.084 | 2.84 |
| T2 | 14 | 1672 | 3.69^{a} | 0.086 | 3.13 |
| SEM | | 55.9 | 0.155 | 0.0081 | 0.231 |
| NC | | 1668 | 3.49 | 0.086 | 2.81 |
| PC | | 1609 | 3.47 | 0.098 | 2.62 |
| T1 | | 1639 | 3.77 | 0.088 | 2.75 |
| T2 | | 1708 | 3.61 | 0.086 | 2.81 |
| | 7 | 1686 | 3.76 | 0.087 | 2.63 |
| | 14 | 1626 | 3.41 | 0.092 | 2.87 |

P-value

| Treatment × Day | 0.8934 | 0.0296 | 0.4626 | 0.2625 |
|---|---|---|---|---|
| Treatment | 0.3383 | 0.0296 | 0.4195 | 0.8287 |
| Day | 0.1315 | 0.0024 | 0.4417 | 0.1479 |

1. Method for increasing laying index, increasing egg mass, and/or increasing percentage of XL eggs of a layer bird (e.g. layer poultry, layer chicken, layer hen), comprising the steps of
a) providing a layer bird;
b) providing a nutritional composition suitable for layer birds comprising an oligosaccharide preparation (e.g. a synthetic oligosaccharide preparation),
wherein the oligosaccharide preparation comprises at least n fractions of oligosaccharides each having a distinct degree of polymerization selected from 1 to n (DP1 to DPn fractions),
wherein n is an integer greater than or equal to 2, and
wherein each fraction comprises from at least about 0.5% to about 90% (e.g. from 1% to 90%;
or e.g. from about 0.5% to about 15%) of anhydro-subunit containing oligosaccharides by relative abundance as determined by mass spectrometry; and
c) administering the nutritional composition to the layer bird.

2. The method according to claim 1, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

3. The method according to claim 1, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

4. The method according to claim 1, wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

5. Egg obtained by the method of claim 1, wherein the egg has a lower risk for comprising Salmonella.

6. Use of an oligosaccharide preparation (e.g. synthetic oligosaccharide preparation) to increase laying index, increase egg mass, and/or increase percentage of XL eggs of a layer bird (e.g. layer poultry, layer chicken, layer hen),
wherein the oligosaccharide preparation comprises at least n fractions of oligosaccharides each having a distinct degree of polymerization selected from 1 to n (DP1 to DPn fractions),
wherein n is an integer greater than or equal to 2; and
wherein each fraction comprises from at least about 0.5% to about 90% (e.g. from 1% to 90%;
or e.g. from about 0.5% to about 15%) of anhydro-subunit containing oligosaccharides by relative abundance as determined by mass spectrometry.

7. Use of an oligosaccharide preparation (e.g. synthetic oligosaccharide preparation) to counteract one or more effects of Salmonella infection, such as a reduction in hen-day egg production, of a layer bird (e.g. layer poultry, layer chicken, layer hen),
wherein the oligosaccharide preparation comprises at least n fractions of oligosaccharides each having a distinct degree of polymerization selected from 1 to n (DP1 to DPn fractions),
wherein n is an integer greater than or equal to 2; and
wherein each fraction comprises from at least about 0.5% to about 90% (e.g. from 1% to 90%;
or e.g. from about 0.5% to about 15%) of anhydro-subunit containing oligosaccharides by relative abundance as determined by mass spectrometry.

8. The use according to claim 6, wherein the oligosaccharide preparation is administered to the bird before, after, and/or simultaneously with the diet of the layer bird, preferably simultaneously with the diet.

9. The use according to claim 6, wherein the oligosaccharide preparation is comprised in a nutritional composition administered to the layer bird.

10. The use according to claim 9, wherein the oligosaccharide preparation is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed).

11. The use according to claim 6, wherein the layer bird is layer poultry (e.g. chicken, duck, goose, turkey, guinea fowl, pigeon, quail), preferably layer chicken.

12. The use according to claim 6, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

13. The use according to claim 6, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

14. The use according to claim 6, wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

15. Oligosaccharide preparation for use in treatment, amelioration and/or prophylaxis of a layer bird suffering from enteric stress caused by Salmonella, wherein the oligosaccharide preparation comprises at least n fractions of oligosaccharides each having a distinct degree of polymerization selected from 1 to n (DP1 to DPn fractions), wherein n is an integer greater than or equal to 2, and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.
